(19) United States
(12) Patent Application Publication
(10) Pub. No.: US 2015/0320468 A1
(43) Pub. Date: Nov. 12, 2015

(54) IMPLANT SYSTEM AND SECURING ELEMENT FOR AN IMPLANT SYSTEM

(71) Applicant: Aesculap AG, Tuttlingen (DE)

(72) Inventors: Sven Krüger, Trossingen (DE); Fabian Hoefer, Tuttlingen (DE)

(21) Appl. No.: 14/804,459

(22) Filed: Jul. 21, 2015

Related U.S. Application Data
(63) Continuation of application No. PCT/EP2014/050971, filed on Jan. 20, 2014.

(30) Foreign Application Priority Data
Jan. 21, 2013 (DE) 10 2013 100 574.8

(51) Int. Cl. A61B 17/86

(52) U.S. Cl. CPC A61B 17/866; A61B 17/8605

(57) ABSTRACT

A fixing element for an implant system includes at least two fixing elements that are fixable to bone parts or the like and at least one connecting element that is fixable to the at least two fixing elements. The fixing element includes a distal end fixing part having a fixing section and a seating part which is connected to the fixing part. The seating part includes a connecting element seating for accommodating the connecting element and a fixing element for fixing the fixing element in the connecting element seating. The seating part is formed from a seating part material. The seating part includes at least one reinforcement element which is formed from a reinforcement element material that has at least one of a greater stiffness and strength than the seating part material.

### CROSS-REFERENCE TO RELATED APPLICATIONS

This application is a continuation of international application number PCT/EP2014/050971 filed on Jan. 20, 2014 and claims the benefit of German application number 10 2013 100 574.8 filed on Jan. 21, 2013.

The present disclosure relates to the subject matter disclosed in international application number PCT/EP2014/050971 of Jan. 20, 2014 and German application number 10 2013 100 574.8 of Jan. 21, 2013, both of which are incorporated herein by reference in their entirety and for all purposes.

### FIELD OF THE INVENTION

The present invention relates to fixing elements for implant systems comprising at least two fixing elements that are fixable to bone parts or the like and at least one connecting element that is fixable to the at least two fixing elements generally, and more specifically to a fixing element for an implant system comprising at least two fixing elements that are fixable to bone parts or the like and at least one connecting element that is fixable to the at least two fixing elements, wherein the fixing element comprises a distal end fixing part comprising a fixing section and a seating part connected to the fixing part, wherein the seating part comprises a connecting element seating for accommodating the connecting element and a fixing element for fixing the fixing element in the connecting element seating, wherein the seating part is formed from a seating part material.

Furthermore, the present invention relates to implant systems generally, and more specifically to an implant system with at least two fixing elements that are fixable to bone parts or the like and with at least one connecting element that is fixable to the at least two fixing elements.

### BACKGROUND OF THE INVENTION

Fixing elements of the type described hereinabove are known particularly in the form of pedicle screws as parts of implantation systems for stabilizing the spinal column and they may comprise a fork-like head in which a connecting element in the form of a bar is insertible and fixable by means of a locking screw. Large forces are required for fixing such a connecting element in order to ensure the desired stability of the implant system after implantation. These forces are introduced via the locking screw which is supported on the seating part. The ever recurring spreading of the seating part due to the high forces involved represents a problem which can lead in particular to a loss of power when tightening, jamming with instruments as well as other problems, and in the worst case even to the locking screw jumping out of the seating part.

In order to prevent this spreading process, it is known for example to provide threads which comprise undercuts for fixing the locking screw. This is described in U.S. Pat. No. 6,726,689 B2 for example. However, forming undercuts in the threads is complicated and difficult particularly when using hard materials.

### SUMMARY OF THE INVENTION

In a first aspect of the invention, a fixing element for an implant system comprises at least two fixing elements that are fixable to bone parts or the like and at least one connecting element that is fixable to the at least two fixing elements. The fixing element comprises a distal end fixing part comprising a fixing section and a seating part which is connected to the fixing part. The seating part comprises a connecting element seating for accommodating the connecting element and a fixing element for fixing the fixing element in the connecting element seating. The seating part is formed from a seating part material. The seating part comprises at least one reinforcement element which is formed from a reinforcement element material that has a greater stiffness and/or strength than the seating part material.

In a second aspect of the invention, an implant system comprises at least two fixing elements that are fixable to bone parts or the like and at least one connecting element that is fixable to the at least two fixing elements. At least one of the at least two fixing elements is a fixing element comprising a distal end fixing part comprising a fixing section and a seating part which is connected to the fixing part. The seating part comprises a connecting element seating for accommodating the connecting element and a fixing element for fixing the fixing element in the connecting element seating. The seating part is formed from a seating part material. The seating part comprises at least one reinforcement element which is formed from a reinforcement element material that has a greater stiffness and/or strength than the seating part material.

### BRIEF DESCRIPTION OF THE DRAWING FIGURES

The foregoing summary and the following description may be better understood in conjunction with the drawing figures, of which:

FIG. 1: shows a schematic, perspective and partly cut-away view of a fixing part of an implant system; and

FIG. 2: a schematic side view of an implant system with two fixing parts fixed to a spinal column.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a fixing element for an implant system comprising at least two fixing elements that are fixable to bone parts or the like and at least one connecting element that is fixable to the at least two fixing elements, wherein the fixing element comprises a distal end fixing part comprising a fixing section and a seating part which is connected to the fixing part, wherein the seating part comprises a connecting element seating for accommodating the connecting element and a fixing element for fixing the fixing element in the connecting element seating, wherein the seating part is formed from a seating part material, wherein the seating part comprises at least one reinforcement element which is formed from a reinforcement element material that has a greater stiffness and/or strength than the seating part material.

Instead of forming the seating part as a whole from a stiffer material which would make it more difficult to produce special shapes of thread, the solution in accordance with the invention that is being proposed offers the option of realizing simple shapes of thread but nevertheless ensures the requisite stiffness and/or strength of the seating part in order to prevent, in particular, any deformation such as the spreading thereof for example. In particular, one, two, three, four or even more reinforcement elements may be provided.

It is particularly advantageous if the modulus of elasticity of the seating part material is smaller than the modulus of elasticity of the reinforcement element material. Thus overall, one can obtain the desired stiffness and/or strength of the seating part by using a reinforcement element material which has a larger modulus of elasticity.

It is particularly advantageous if the value of the modulus of elasticity of the reinforcement element material amounts to at least 1.5 times the value of the modulus of elasticity of the seating part material. For example, this condition is fulfilled when using a cobalt-chromium steel as the reinforcement element material and also a titanium alloy as the seating part material.

Expediently, the seating part material is transparent to X-rays or is substantially transparent to X-rays. In this way, shadows in the radiographs due to the seating part can be prevented or at least minimized.

Advantageously, the reinforcement element material is opaque to X-rays or is substantially opaque to X-rays. Consequently, the reinforcement element can be used in order to render the positioning of the seating part visible and thereby depict the position of the fixing element as a whole in a radiograph.

Preferably, the seating part material has a smaller allergy potential than the reinforcement element material. This makes it possible in particular to arrange the reinforcement element on the seating part in such a way that it is completely or substantially completely surrounded by the seating part material for example so that contact between the reinforcement element material and the body of the patient can be minimized or even prevented completely.

It is especially advantageous if the seating part material is titanium or contains titanium. Titanium is a bio-inert marker, although its oxide, TiO₂, forms a bio-active marker.

It is expedient if the seating part material is a titanium alloy and in particular Ti6AL4V or Ti6AL7Nb. In practice, these materials have already proved their worth as implant materials.

In a further preferred embodiment of the invention, provision may be made for the reinforcement element material to be a steel and/or contain cobalt and/or chromium and/or be a ceramic or contain a ceramic. In the case of such reinforcement element materials, it is possible to produce reinforcement elements having the requisite stiffness and/or strength.

Furthermore, it is expedient if the reinforcement element material is a cobalt-chromium alloy or a cobalt-chromium steel. These materials, which do of course have a raised allergy potential and can trigger allergic reactions, do however have the requisite stability for forming implants. For the purposes of preventing allergic reactions, they can be encapsulated particularly in biocompatible materials or provided with biocompatible coatings in order to prevent especially unwanted leakage of cobalt and chromium into the body of a patient.

It is advantageous if, for the purposes of forming the connecting element seating, the seating part has a substantially U-shaped longitudinal section with two free limbs extending away from the fixing part in the proximal direction. Such a construction of the seating part enables a connecting element to be inserted from above. Thus overall, the seating part can be constructed in the form of a fork-like head with a fixing screw as the fixing element for example.

It is particularly expedient if the seating part is in the form of a seating part wall section in the region of the free limbs where the cross-sectional thickness thereof is a relative or absolute minimum. In other words, this means for example that the free limbs can be thicker towards the lateral edges or ends thereof than approximately in the central area between the lateral edges where the thickness is thus at least a relative minimum and in some circumstances is an absolute minimum. This, for example, enables an appropriate thread to be provided for the purposes of screwing-in a fixing or locking screw, but at least one reinforcement element can additionally be arranged in the regions of thicker wall thickness in order to achieve the stability desired for the seating part. One, two, three or even more reinforcement elements may be provided on each limb in dependence on the degree of stability required and the amount of space that is available on the limbs.

The overall stability of the fixing element can be increased if the fixing part and the seating part are connected to one another such as to be immovable. For example, they could be formed in one piece manner or soldered or welded to one another in order to prevent practically any relative movement between the fixing part and the seating part.

It can however also be expedient if the fixing part is adjustable relative to the seating part in an adjusting position and is fixable relative to the seating part in a fixing position. For example, the fixing element could thus be in the form of a polyaxial screw which enables the fixing part to be inserted into a bone with an optimized orientation whereafter the seating part is aligned relative to the fixing part in such a way that a connecting element can be inserted in the optimal manner with the desired alignment and then fixed immovably to the fixing element.

A variably orientable fixing element can be constructed in a particularly simply manner if the fixing part and the seating part are connected to one another in articulated manner. Basically here, every type of articulated joint is conceivable.

It is advantageous if an articulated joint is provided between a proximal end of the fixing part and a distal end of the seating part and if the seating part comprises at the distal end thereof a seating for the proximal end of the fixing part. Such an arrangement also enables an articulated coupling to be provided between the seating part and the fixing part.

In order to enable almost any desired orientation of the seating part relative to the fixing part, it is expedient if the articulated joint is in the form of a ball joint connection and if the proximal end of the fixing part is in the form of a spherical segment head and the seating is in the form of a dome shaped seat. This permits, in particular, free rotation of the seating part relative to the fixing part about a central point of the head.

The process of fixing a connecting element in or on the seating part can be further simplified if the seating part incorporates a fixing thread which cooperates with a fixing element thread of the fixing element.

Expediently, the fixing thread is formed in the region of the free limbs. This enables a connecting element to protrude laterally from the seating part when it is affixed thereto by a fixing element which is screwed into the seating part from above.

In particular, the stability of the fixing element can be increased in that the seating part is formed in one piece manner without the at least one reinforcement element. For example, the seating part can then be manufactured in a casting process.

In order to facilitate the placement of the at least one reinforcement element on the seating part, it is expedient if the seating part comprises at least one reinforcement element seating into which the at least one reinforcement element is inserted.

Hereby for example, this may be a laterally open groove on the seating part into which the reinforcement element is inserted and then fixed such as by adhesion, soldering or welding for example. It is also conceivable for the reinforcement element to be cast in the reinforcement element seating thereby producing the seating part in a two-stage casting process.

In accordance with a further preferred embodiment of the invention, provision may be made for the at least one reinforcement element to be fixed in the at least one reinforcement element seating in force-locking and/or positively locking manner. In this way, one can prevent the reinforcement element from being able to move relative to the seating part and in particular, such that it cannot escape therefrom in an unwanted manner.

The at least one reinforcement element can be fixed to the seating part in a particularly simple and secure manner if it is held in the at least one reinforcement element seating by means of a press fit. The reinforcement element can thus be produced a little oversized and then be pressed into the reinforcement element seating provided in the seating part.

The at least one reinforcement element seating can be produced in a simple manner if it is in the form of a through opening or a blind hole.

Expediently, the at least one reinforcement element seating is in the form of a boring. A boring can be easily produced, namely in the form of a through-hole as well as a blind hole boring.

The production of the fixing element can be further simplified if the at least one reinforcement element is in the form of a rod or substantially in the form of a rod. For example, reinforcement elements can then be manufactured by cutting off pieces from a continuous length of material or a profiled material. In principle thereby, the cross-sectional shape of the reinforcement element can be selected at will. Advantageously, the reinforcement element has a cross-sectional shape which, for example, exhibits greater flexural stability in comparison to a round bar profile, i.e. a reinforcement element practically in the form of a bending-resistant profile.

Preferably the at least one reinforcement element has a circular or substantially circular cross section. Such a reinforcement element can be easily and securely inserted into a reinforcement element seating in the form of a boring such as to be a snug-fit, by being pressed-in for example if the diameter of the reinforcement element is only a little larger than the inner diameter of the reinforcement element seating.

In accordance with a further preferred embodiment of the invention, provision may be made for the at least one reinforcement element seating to define a longitudinal axis which is aligned in parallel with or substantially in parallel with a longitudinal axis defined by the seating part and/or with a longitudinal axis defined by the fixing part. For example, seating part wall sections of minimum thickness can thus be formed whereby it can nevertheless be ensured that the reinforcement elements can be completely accommodated in the seating part. Moreover, this arrangement has the advantage that when using a reinforcement element material that is opaque to X-rays for example, the orientation of the seating part and/or that of the fixing part is recognizable in a radiograph.

It is expedient, if the at least one reinforcement element seating in the free limbs is formed in the region thereof or adjacent thereto. This enables the at least one reinforcement element that is inserted into the at least one reinforcement element seating to increase the stability of the seating part at precisely that point where it is needed, namely for example, within the region of the free limbs.

In order to prevent damage to the seating part particularly in the region of the free limbs, it is advantageous if the at least one reinforcement element seating is arranged or formed in a region of the free limbs where they are of greater thickness than in the region of minimum wall thickness. In other words, the at least one reinforcement element seating is preferably formed at the point where more material is available so that, after the at least one reinforcement element seating has been formed, the stability of the seating part will not be endangered but rather will be increased in the desired manner by the insertion of the at least one reinforcement element.

Different thicknesses in the region of the free limbs can be achieved in a simple manner especially if the cross section of an outer contour defined by the seating part is not round. Preferably, the cross section of the outer contour is oval or rectangular and in particular is rectangular with rounded-off corners. Free limbs with varying thicknesses can thus be formed, these being particularly suitable for forming reinforcement element seatings.

It is preferable that the reinforcement element material does not form an outer surface of the seating part. This has the particular advantage that in principle, materials which have a certain allergy potential could also be used as the material for the reinforcement element because they cannot come directly into contact with the body tissue of a patient. This can be achieved in particular by surrounding the reinforcement element material with a suitable coating or otherwise encapsulating or enclosing it.

Furthermore, it can also be advantageous if the at least one reinforcement element seating is sealed off. For example, it can be sealed by caulking a flange. Due to the sealing of the reinforcement element seating for example, the process of coating the at least one reinforcement element when it is made of a reinforcement element material of higher allergy potential becomes redundant since it is quasi encapsulated therewith and therein in the course of the process of sealing off the at least one connecting element seating.

In accordance with a further preferred embodiment of the invention, provision may be made for at least one closure element for sealing off the at least one reinforcement element seating. With the aid of the at least one closure element, the at least one reinforcement element seating can be sealed off in a simple manner. If the reinforcement element seating comprises two or more openings, then preferably two or more closure elements are also provided which preferably seal off the reinforcement element seating in gas-tight and liquid-tight manner.

The at least one reinforcement element seating can be sealed off in a particularly simple and secure way if the closure element is in the form of a headless screw or a cover that is fixed in the at least one reinforcement element seating in force-locking and/or positively locking manner. Thus, for the purposes of sealing the at least one reinforcement element seating, the headless screw can be screwed-in or the cover can be pressed-in or fixed with adhesive for example.

For the purposes of fixing a connecting element to the fixing element, it is expedient if the fixing thread is an internal thread or an external thread and if the fixing element is in the form of a screw or a nut. The provision of an internal thread in particular enables the fixing element to be of particularly compact construction.

It is particularly advantageous, if the fixing part is in the form of a bone screw or a bone hook. The fixing element can then be securely anchored in bones or bone tissue by the fixing part.

The present invention further relates to an implant system comprising at least two fixing elements that are fixable to bone parts or the like and at least one connecting element that is fixable to the at least two fixing elements, wherein at least one of the at least two fixing elements is a fixing element comprising a distal end fixing part comprising a fixing section and a seating part which is connected to the fixing part, wherein the seating part comprises a connecting element seating for accommodating the connecting element and a fixing element for fixing the fixing element in the connecting element seating, wherein the seating part is formed from a seating part material, wherein the seating part comprises at least one reinforcement element which is formed from a reinforcement element material that has a greater stiffness and/or strength than the seating part material.

Thus, the overall stability of the implant system can be increased overall in a simple and secure manner.

The implant system can be adapted individually to the physiology of a patient in a simple manner if the connecting element is a bar or a plate comprising a section in the form of a bar. In dependence on the amount of space available as well as the stability demanded, then either a straight or curved or individually bent rod and/or too, a suitable plate can be used for stabilizing the spinal column of a patient for example.

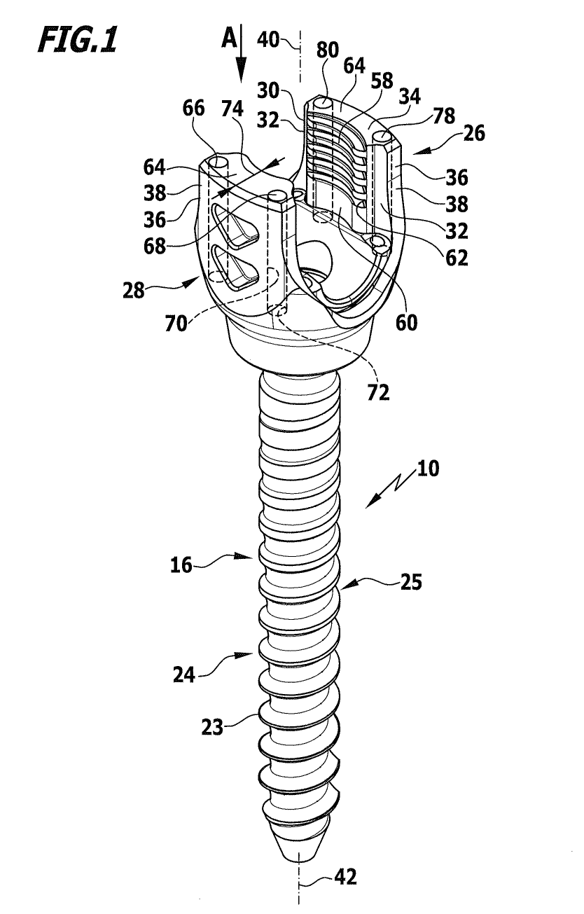

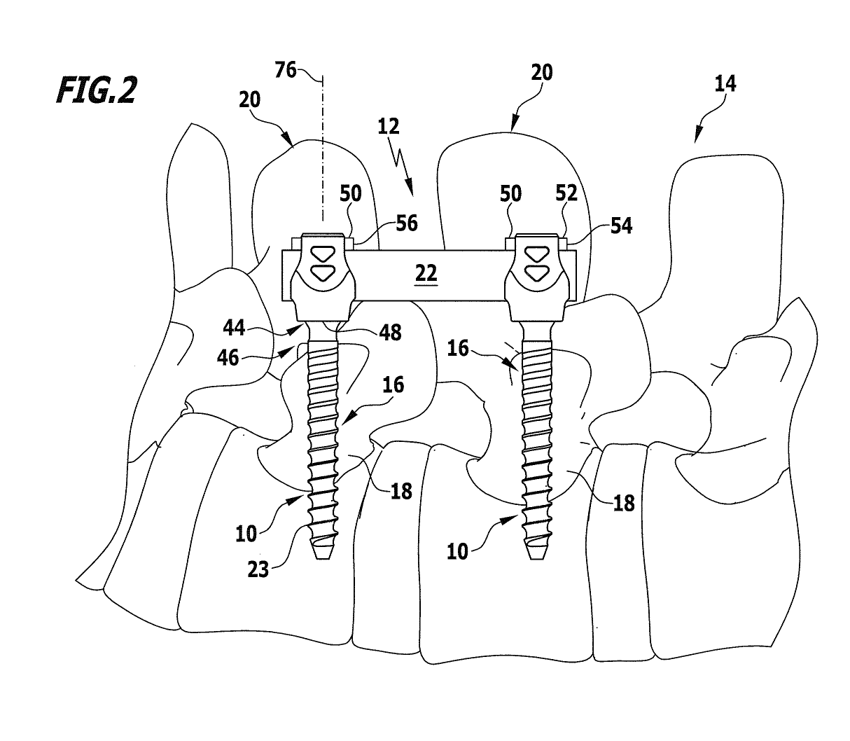

A fixing element 10 of an implant system 12 for the stabilization of a spinal column 14 that is schematically illustrated in FIG. 2 is schematically illustrated in FIG. 1. Two fixing elements 10 in the form of bone screws 16 that are preferably formed by pedicle screws which are used for screwing into pedicles 18 of the vertebrae 20 of the spinal column 14 are illustrated in FIG. 2. Furthermore, the implant system 12 incorporates a connecting element 22 which can be in the form of a bar, whether straight or curved, or in the form of a plate comprising a section in the form of a bar.

The fixing element comprises a fixing part 24 and a seating part 26 which is adjoined to the proximal-side of the fixing part 24. The fixing part 24 comprises a fixing section 25 incorporating a bone thread 23 so that the fixing part 24 can be screwed into bone tissue. Alternatively, the fixing part 24 could also be in the form of a bone hook or a bone nail.

The seating part 24 is in the form of a fork-like head 28 which comprises a connecting element seating 30 that is defined by two diametrically opposed recesses 32 in a sleeve-shaped wall 34 of the seating part 26 oriented in the proximal direction. The connecting element seating 30 is thus bounded laterally by two diametrically opposed free limbs 36 which are oriented in the proximal direction and form the seating part sections 38.

The seating part 26 defines a longitudinal axis 40 which is in alignment with a longitudinal axis 42 of the fixing part 24 in the case of a fixing element 10 having a seating part 26 which is connected immovably to the fixing part 24. As an option, the seating part 26 could also be articulated to the fixing part 24, for which purpose provision may be made for an articulated joint 44 that is in the form of a ball joint for example. In this case, a proximal end 48 of the fixing part 24 is in the form of a head having the shape of a segment of a sphere which is formed so as to correspond to a seating in the form of a dome shaped seat in the region of a distal end of the seating part 26.

For the purposes of fixing the connecting element 22 in the connecting element seating 30, there serves a fixing element 50 which can be in the form of a headless screw 52 having a fixing element thread 54 in the form of an external thread 56 for example. The fixing element thread 54 corresponds to a fixing thread 58 of the seating part 26. The fixing thread 58 is formed on mutually facing inner surfaces 60 of the seating part wall sections 38 so that each of these seating part wall sections 38 comprises a plurality of thread grooves 62 extending over an angular range of about 80°.

When screwing the fixing element 50 into the seating part 26 for the purposes of clamping the connecting element 22 to the respective fixing element 10, unwanted spreading of the free limbs 36 can occur when using conventional screws whereby the free ends 64 thereof move somewhat away from the longitudinal axis 40 in the radial direction. In order to prevent this, two mutually parallel reinforcement element seatings 66 that are aligned in parallel with the longitudinal axis 40 are formed on each seating part wall section 38 for the purposes of accommodating a respective reinforcement element 68. The reinforcement element seating 66 could optionally be in the form of a through opening or a blind hole 70. In particular, a boring 72 can be produced for this purpose. A reinforcement element 68 is inserted into each reinforcement element seating 66. This element is in the form of a rod and preferably has a circular or substantially circular cross section. In the exemplary embodiment of the fixing element 10 illustrated schematically in FIG. 1, the reinforcement element 68 is in the form of a cylindrical rod.

The reinforcement element seatings 66 are thus formed in the free limbs 36 or in the vicinity thereof. Furthermore, they are arranged in a region of the seating part wall sections 38 having a defined thickness 74 in the radial direction which is greater than in the region of a plane of symmetry 76 of the seating part 26 containing the longitudinal axis 40. The thickness 74 of the seating part wall sections within this region is at a relative minimum.

The reinforcement elements 68 are held in force-locking and/or positively locking manner in the reinforcement element seatings 66 so as to prevent them falling out unintentionally. In order to prevent the reinforcement elements 68 from contacting the body tissue of the patient, each reinforcement element seating 66 is sealed off by a closure element 78 so that the reinforcement elements 68 are practically encapsulated in the seating part wall sections 38. In practice therefore, the closure element 78 forms a cover 80 for the reinforcement element seating 66. If the latter has more than one opening, then a corresponding number of covers 80 is preferably provided. Alternatively, the reinforcement elements 68 can also be provided with a coating so that they are already totally encapsulated before being inserted into the reinforcement element seatings 66.

For the purposes of optimizing a function of the reinforcement elements 68, namely, for strengthening or reinforcing the seating part wall sections 38, the reinforcement elements 68 are formed from a reinforcement element material which exhibits greater stiffness and/or strength than the seating part material from which the seating part 26 is formed. Preferably, the modulus of elasticity of the reinforcement element material is greater than the modulus of elasticity of the seating part material. In particular, it is advantageous if a value of the modulus of elasticity of the reinforcement element material amounts to at least 1.5 times the value of the modulus of elasticity of the seating part material.

Preferably, the seating part materials used for forming the seating parts 26 of the fixing elements 10 are transparent to X-rays or are substantially transparent to X-rays. In contrast thereto, the reinforcement element material in particular could also be opaque to X-rays or substantially opaque to X-rays. Consequently, the reinforcement elements 68 can be made visible in a radiograph and thus too the orientation of the seating part 26 can be depicted.

The described encapsulation of the reinforcement elements 68 or a coating thereon is especially advantageous if the reinforcement element material has a certain allergy potential. Contact of the allergy-potential material with body tissue of the patient can thereby be prevented.

It is expedient to use titanium as the seating part material. However, apart from titanium, it can also contain further elements if, for example, it is in the form of a titanium alloy such as Ti6AL4V or Ti6AL7Nb for example.

Suitable in particular as reinforcement element materials are steels such as steels containing cobalt and chromium for example, and in particular the cobalt-chromium based alloys that are frequently used for medical products.

The described construction of the fixing element 10 makes it possible to use a seating part material which is relatively soft so that the fixing thread 58 can be worked into the free limbs 36 in a simple manner. The reinforcement elements 68 serve to prevent the free limbs 36 from deforming and/or spreading-out during the process of screwing-in the fixing element 50 since they lend a degree of stiffness and/or strength to the seating part 26 such as would only be attainable in practice if the material from which the reinforcement elements 68 are manufactured is likewise used as the seating part material.

In order to provide sufficient material on the free limbs 36 as would enable the reinforcement element seatings 66 to be formed, the cross section of the seating part 26 in plan view in the distal direction, i.e. in the direction of the arrow A in FIG. 1 has a non-round outer contour and in particular it has an oval or rectangular contour, preferably a rectangular outer contour with rounded-off corners.

What is claimed is:

1. A fixing element for an implant system comprising at least two fixing elements that are fixable to bone parts or the like and at least one connecting element that is fixable to the at least two fixing elements, wherein the fixing element comprises a distal end fixing part comprising a fixing section and a seating part which is connected to the fixing part, wherein the seating part comprises a connecting element seating for accommodating the connecting element and a fixing element for fixing the fixing element in the connecting element seating, wherein the seating part is formed from a seating part material, wherein the seating part comprises at least one reinforcement element which is formed from a reinforcement element material that has at least one of a greater stiffness and strength than the seating part material.

2. The fixing element in accordance with claim 1, wherein a modulus of elasticity of the seating part material is smaller than a modulus of elasticity of the reinforcement element material.

3. The fixing element in accordance with claim 1, wherein the seating part material is transparent to X-rays or is substantially transparent to X-rays.

4. The fixing element in accordance with claim 1, wherein at least one of the reinforcement element material is opaque to X-rays or is substantially opaque to X-rays and the seating part material has a lower allergy potential than the reinforcement element material and the seating part material is titanium or contains titanium.

5. The fixing element in accordance with claim 1, wherein the reinforcement element material at least one of is a steel and contains cobalt and chromium and is a ceramic or contains a ceramic.

6. The fixing element in accordance with claim 1 wherein, for the purposes of forming the connecting element seating, the seating part has a substantially U-shaped longitudinal section with two free limbs extending away from the fixing part in the proximal direction.

7. The fixing element in accordance with claim 6, wherein the seating part is in the form of a seating part wall section in the region of the free limbs and the thickness thereof in cross section is at a relative or absolute minimum.

8. The fixing element in accordance with claim 1, wherein the fixing part and the seating part are connected immovably to one another.

9. The fixing element in accordance with claim 1, wherein the fixing part is adjustable relative to the seating part in an adjusting position and is fixable relative to the seating part in a fixing position.

10. The fixing element in accordance with claim 1, wherein the seating part comprises a fixing thread which cooperates with a fixing element thread of the fixing element.

11. The fixing element in accordance with claim 1, wherein the seating part is formed in one piece without the at least one reinforcement element.

12. The fixing element in accordance with claim 1, wherein the seating part comprises at least one reinforcement element seating into which the at least one reinforcement element is inserted.

13. The fixing element in accordance with claim 12, wherein the at least one reinforcement element seating is in the form of a through opening or a blind hole.

14. The fixing element in accordance with claim 1, wherein the at least one reinforcement element is in the form of a rod or substantially in the form of a rod and has a circular or substantially circular cross section.

15. The fixing element in accordance with claim 12, wherein at least one of
the at least one reinforcement element seating defines a longitudinal axis which is aligned in parallel with or substantially in parallel with at least one of a longitudinal axis defined by the seating part and with a longitudinal axis defined by the fixing part and
the at least one reinforcement element seating is formed in the free limbs or in the region thereof or adjacent thereto and
the at least one reinforcement element seating is arranged or formed in a region of the free limbs that is of greater thickness than in the region of minimum wall thickness.

16. The fixing element in accordance with claim 1, wherein at least one of an outer contour defined by the cross section of the seating part is not round and in particular, is oval or rectangular with preferably rounded-off corners and the reinforcement element material does not form an outer surface of the seating part.

17. The fixing element in accordance with claim 12, wherein at least one of the at least one reinforcement element seating is sealed off particularly by caulking a flange, and the fixing element comprises at least one closure element for sealing off the at least one reinforcement element seating.

18. The fixing element in accordance with claim 1, wherein the fixing part is in the form of a bone screw or a bone hook.

19. An implant system comprising at least two fixing elements that are fixable to bone parts or the like and at least one connecting element that is fixable to the at least two fixing elements, wherein at least one of the at least two fixing elements is a fixing element comprising a distal end fixing part comprising a fixing section and a seating part which is connected to the fixing part, wherein the seating part comprises a connecting element seating for accommodating the connecting element and a fixing element for fixing the fixing element in the connecting element seating, wherein the seating part is formed from a seating part material, wherein the seating part comprises at least one reinforcement element which is formed from a reinforcement element material that at least one of has a greater stiffness and strength than the seating part material.

20. The Implant system in accordance with claim 19, wherein the connecting element is a bar or a plate comprising a section in the form of a bar.
